(19) United States
(12) Patent Application Publication
(10) Pub. No.: US 2011/0255762 A1
(43) Pub. Date: Oct. 20, 2011

(54) METHOD AND SYSTEM FOR DETERMINING A REGION OF INTEREST IN ULTRASOUND DATA

(76) Inventors: Harald Deischinger, Frankenmarkt (AT); Otmar Scherzer, Klosterneuburg (AT); Andreas Obereder, Adlwang (AT)

(21) Appl. No.: 12/761,279

(22) Filed: Apr. 15, 2010

(51) Int. Cl. G06T 7/00; A61B 8/14

(52) U.S. Cl. 382/131; 600/443

(57) ABSTRACT

Methods and systems for determining a region of interest in ultrasound data are provided. One method includes defining an ROI within an acquired ultrasound data set and identifying a plurality of different image planes within the acquired ultrasound data set. The method further includes determining a significant edge from at least one border of the ROI based on the plurality of image planes and adjusting the ROI based on the determined significant edge.

### BACKGROUND OF THE INVENTION

The subject matter disclosed herein relates generally to ultrasound imaging systems, and more particularly to methods for determining a region of interest in ultrasound images.

Ultrasound imaging systems typically include ultrasound scanning devices, such as ultrasound probes having transducers that are connected to an ultrasound system to control the acquisition of ultrasound data for performing various ultrasound scans (e.g., imaging a volume or body). The ultrasound system usually includes a control portion (e.g., a control console or portable unit) that provides interfaces for interacting with a user, such as receiving user inputs and displaying acquired ultrasound images.

Conventional ultrasound systems allow a user to define a region of interest (ROI) within an acquired volume data set for further processing, such as to generate a three-dimensional (3D) image from a plurality of two-dimensional (2D) image slices. For example, in fetal ultrasound applications, the ROI may be the face of the fetus. Because of the surrounding fluid, such as amniotic fluid, and the surrounding uterine tissue, the ROI may be have to readjusted numerous times in order to properly render the face of the fetus in the 3D image such that the entire face is visible in the 3D image. Inexperienced ultrasound users may have significant difficulty in defining the ROI to obtain the proper visualization and experienced users still must take the time to move and readjust the ROI. Accordingly, defining the ROI to obtain the proper visualization for subsequent processing (such that the area of interest is not obstructed) can be a time consuming and difficult process.

### BRIEF DESCRIPTION OF THE INVENTION

In accordance with various embodiments, a method for modifying a region of interest (ROI) in an ultrasound data set is provided. The method includes defining an ROI within an acquired ultrasound data set and identifying a plurality of different image planes within the acquired ultrasound data set. The method further includes determining a significant edge from at least one border of the ROI based on the plurality of image planes and adjusting the ROI based on the determined significant edge.

In accordance with other various embodiments, a method for adjusting a region of interest (ROI) in an ultrasound data set is provided. The method includes determining an ROI based on an ROI box defined within at least two image planes, wherein the ROI box has a width, height and depth. The method further includes identifying pixels from a top side of the ROI box that define a border wherein pixels change from tissue pixels to fluid pixels and fitting a curve to a contour based on the border. The method also includes adjusting the height of the ROI box based on the fitted curve.

In accordance with yet other various embodiments, an ultrasound system is provided that includes an ultrasound probe for acquiring ultrasound data for an object of interest and a user interface for defining a region of interest (ROI) within at least two different image planes within the ultrasound data. The method further includes an ROI defining module configured to adjust an ROI based on a determination of a significant edge from at least one border of the ROI based on the two image planes.

### BRIEF DESCRIPTION OF THE DRAWINGS

FIG. 1 is a flowchart of a method for defining a region of interest (ROI) within an ultrasound data in accordance with various embodiments.

FIG. 2 is a screenshot illustrating a rendered image having tissue obstructing a portion of the image.

FIG. 3 is a screenshot illustrating an image plane corresponding to an image slice.

FIG. 4 is a screenshot illustrating an image plane corresponding to another image slice.

FIG. 5 is a screenshot illustrating an image plane corresponding to another image slice.

FIG. 6 is an image illustrating a contour line determined in accordance with various embodiments.

FIG. 7 is another image illustrating a contour line determined in accordance with various embodiments.

FIG. 8 is a screenshot illustrating an adjusted ROI in accordance with various embodiments and the corresponding rendered image.

FIG. 9 is a block diagram of a diagnostic imaging system including an ROI defining module in accordance with various embodiments.

FIG. 10 is a block diagram of an ultrasound processor module of the diagnostic imaging system of FIG. 9 formed in accordance with various embodiments.

FIG. 11 is a diagram illustrating a 3D capable miniaturized ultrasound system in which various embodiments may be implemented.

FIG. 12 is a diagram illustrating a 3D capable hand carried or pocket-sized ultrasound imaging system in which various embodiments may be implemented.

FIG. 13 is a diagram illustrating a 3D capable console type ultrasound imaging system in which various embodiments may be implemented.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide a system and method for defining or adjusting a region of interest (ROI) in an ultrasound data set. For example, by practicing at least one of the embodiments, an ROI is automatically adjusted for rendering an image thereof, which may include automatically adjusting the ROI to remove fluid or tissue obstructing the view to an object of interest (e.g., a fetus). A technical effect of at least one embodiment is the automatic identification of an ROI, which may be subsequently rendered, thereby reducing the amount of time adjusting the ROI, such as the height and curvature of the ROI. Additionally, by practicing at least one embodiment, the technical skill of the ultrasound system user needed to adjust the ROI is also reduced.

Accordingly, various embodiments define or identify an ROI automatically using a plurality of image planes from a volume of interest in an ultrasound data set. Although the various embodiments are described in connection with defining and adjusting an ROI wherein the object of interest is a fetus, the various embodiments may be implemented in connection with different ultrasound imaging applications, as well as other imaging modalities, for example, computed tomography (CT) imaging or magnetic resonance (MR) imaging.

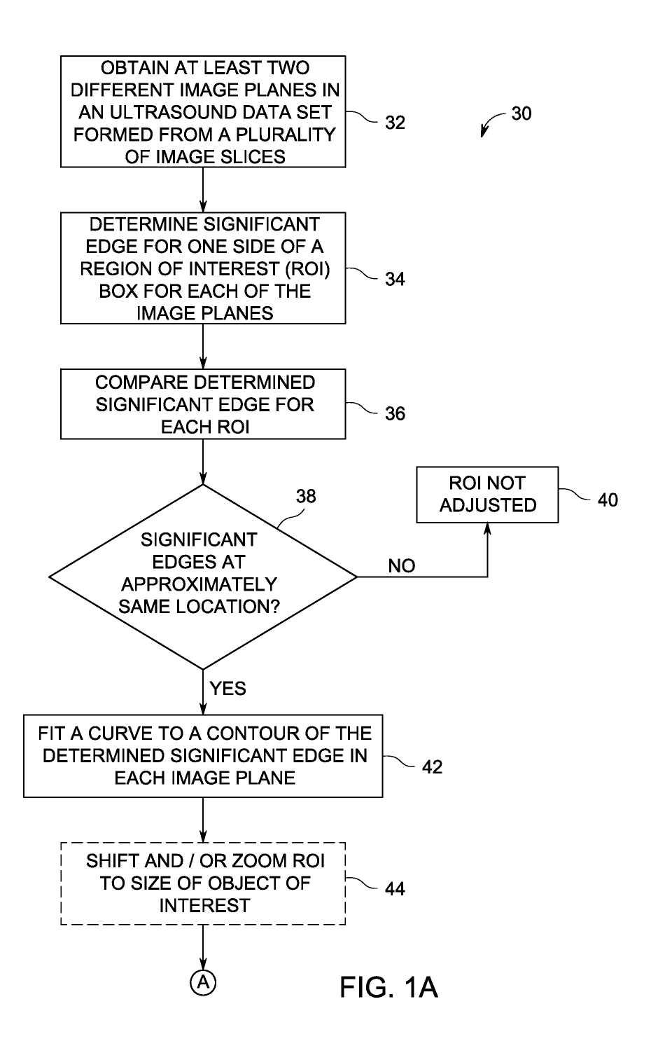

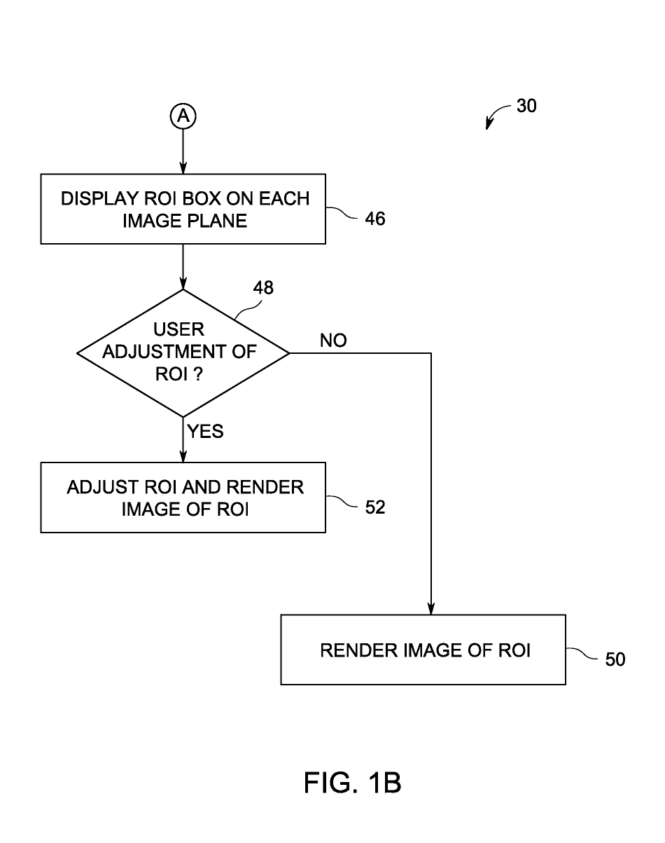

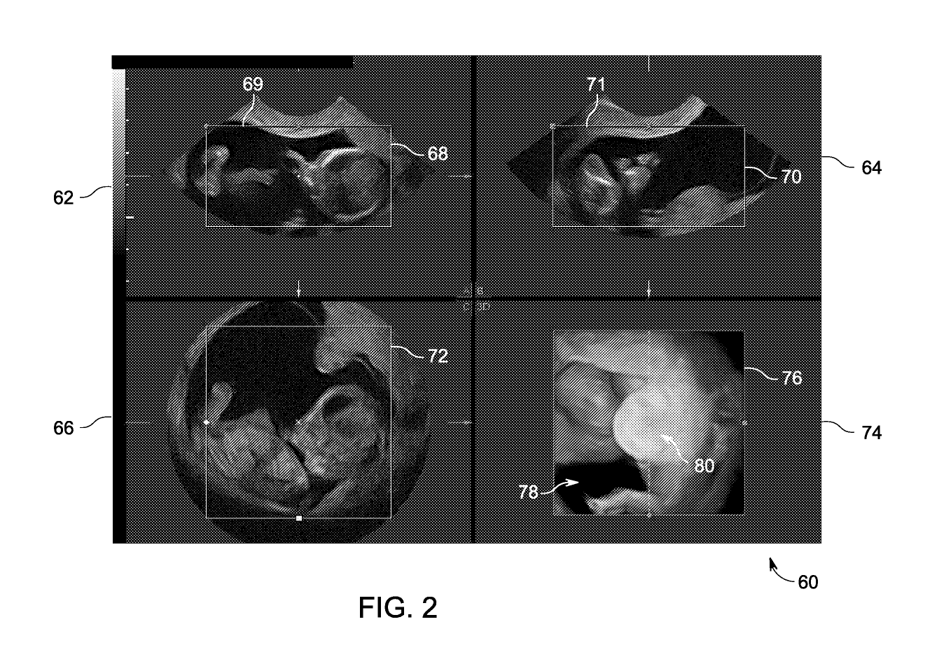

One embodiment of a method 30 for defining an ROI within an ultrasound data set is shown in FIG. 1. The method 30 automatically adjusts the ROI for rendering an image thereof such that, for example, tissue obstructing the view of an object of interest in removed from the ROI. For example, FIG. 2 is a screenshot 60, which may form a portion of or all of a display of an ultrasound image. The screenshot 60 illustrates three image planes 62, 64 and 66 in each of three quadrants of the display. The illustrated image planes 62, 64 and 66 correspond to arbitrary or selected image planes in an ultrasound image data set of an imaged fetus. The image planes 62, 64 and 66 (also identified as images plane A, B and C) generally correspond, respectively, to an image aligned with the axis of the ultrasound probe that acquired the image (Image Plane A), an image orthogonal to Image Plane A (Image Plane B), and a coronal image (Image Plane C) that is orthogonal to both Image Planes A and B and generally parallel to the scanning surface of the ultrasound probe.

Each of the image planes 62, 64 and 66 is shown with an ROI defining portion, illustrated as an ROI box 68, 70 and 72, respectively, defining an ROI (e.g., a portion of the imaged fetus) in each image slice. It should be noted that the ROI box 68, 70 and 72 defines the same ROI of the object of interest from different planes. The ROI box 68, 70 and 72 illustrated in FIG. 2 may be positioned manually by a user, for example, in one of the image views corresponding to one of the image planes 62, 64 and/or 66 or may be determined, for example, based on identification of landmarks within the image, such as using a template or matching process, which may include a contour detection process for a target object (e.g., a fetus). Also, the ROI may be defined by different shaped elements and is not limited to a box. Thus, the ROI box may be defined by a square or rectangular region, or other shaped regions. The ROI box is generally defined by a width, height and depth as described in more detail herein.

The image 74 is a rendered image of the ROI defined by the ROI box 68, 70 and 72, which corresponds to ROI box 76. As can be seen in the 3D rendered image of a fetus 78, a portion of the fetus 78, which may include a particular area of interest, in this case the face of the fetus 78, is obstructed by rendered tissue 80. Accordingly, after viewing the rendered image 74, a user would need to adjust the ROI by adjusting the size or curvature of an edge of the ROI box 68, 70 or 72.

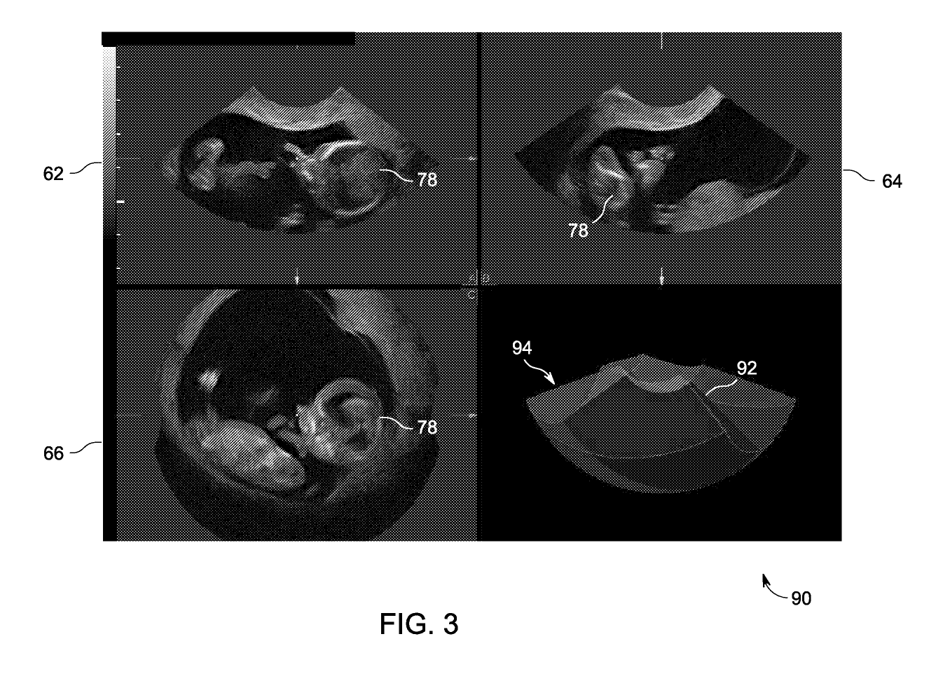

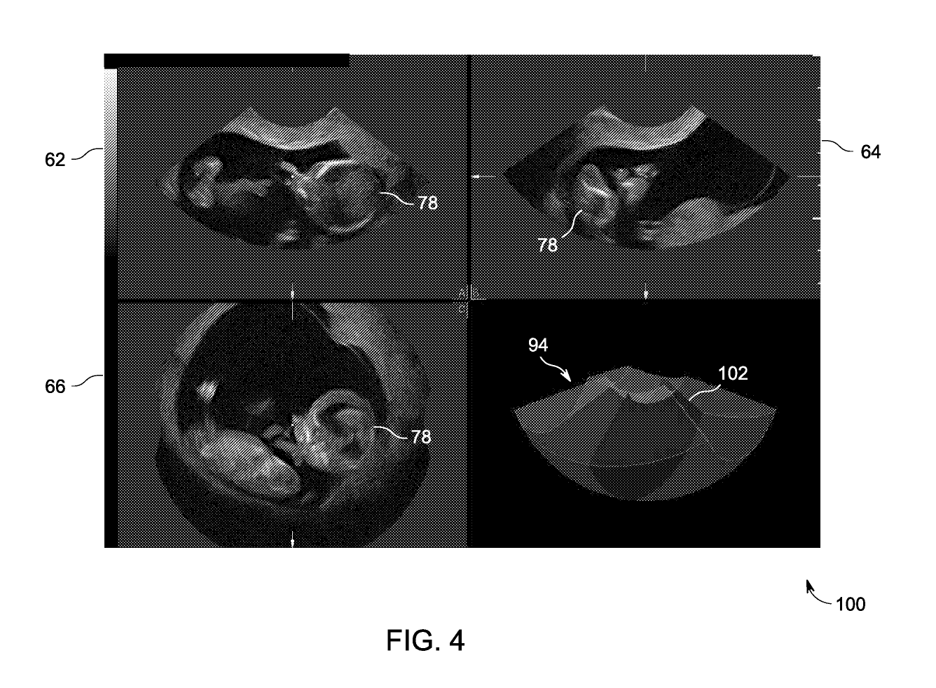

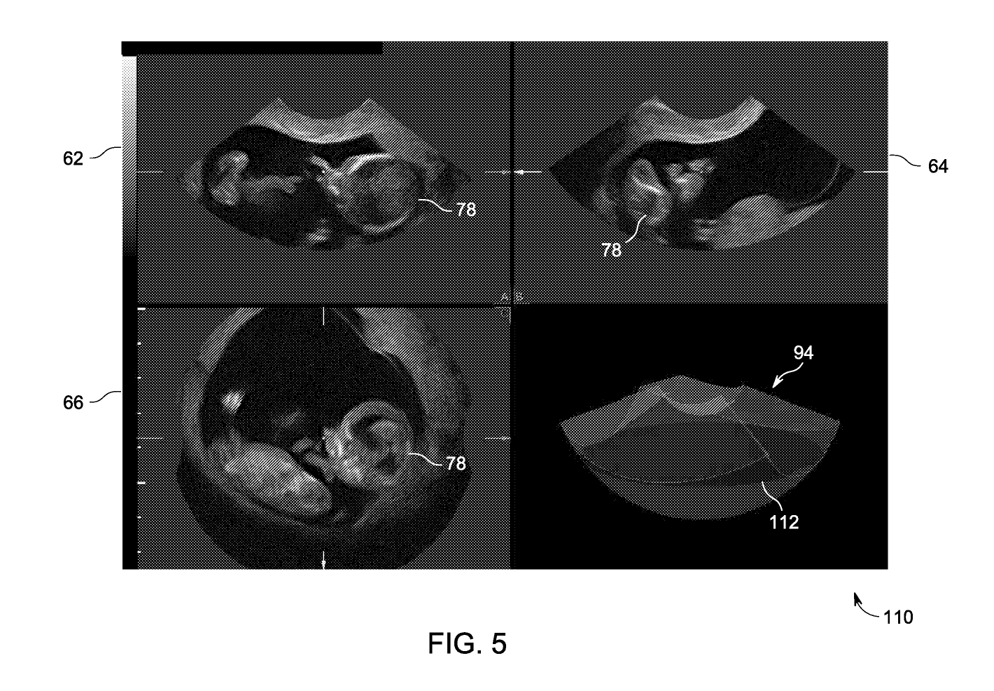

Accordingly, the rendered image 74 is based on an ROI defined using a plurality of image planes as generally illustrated in the screenshots 90, 100 and 110 of FIGS. 3 through 5, wherein like numerals represent like parts throughout the Figures. FIG. 3 illustrates a plane 92 within the image volume 94 (which in the illustrated embodiment is the fetus 78) corresponding to the image plane (Image Plane A) 62. Likewise, FIG. 4 illustrates a plane 102 within the image volume 94 corresponding to the image plane (Image Plane B) 64. Additionally, FIG. 5 illustrates a plane 112 within the image volume 94 corresponding to the image plane (Image Plane C) 66. It should be noted that the image volume 94 is shown for illustrative purposes and is not necessarily displayed to the user.

The image planes 62, 64 and/or 66 in the illustrated embodiment correspond to the orientations of image plane 92 aligned with the axis of the ultrasound probe, image plane 102 that is orthogonal to image plane 92 and image plane 112 that is orthogonal to both image planes 92 and 102, as well as parallel to the scanning surface of the ultrasound probe within the imaged volume. However, the image planes may be any one of a plurality of different image planes 62, 64 and/or 66 of the volume 94 and are not limited to the orientations illustrated by image planes 92, 102 and 112 shown. Accordingly, one or more of the image planes 62, 64 and/or 66 may be oriented differently within the volume 94 and defined by different image views. Additionally, the various embodiments may adjust or define the ROI using more or less than three image planes, such as two or four image planes.

Accordingly, the method 30 of FIG. 1 includes obtaining or selecting image plane data at 32. For example, at least two image planes corresponding to two different image planes in an ultrasound data set are obtained, which may include accessing stored ultrasound data, such as a 3D data set of an object of interest, or acquiring ultrasound data by scanning a patient and obtaining the data while the patient is being scanned or during the patient examination, but not necessarily while the patient is being scanned. The image plane data may correspond, for example, to one or more of the image planes 62, 64 and/or 66 illustrated in FIGS. 3 through 5. In some embodiments, the image plane data includes two image planes that are orthogonal to one another.

It should be noted that the ultrasound system in various embodiments acquires image slices in a fan-shaped geometry to form a volume, which geometrically is typically a section of a torus. When reference is made herein to obtaining or selecting image planes in the various embodiments, this generally refers to selecting one or more arbitrary image planes from an acquired volume, for example, an acquired 3D ultrasound data set.

After the image planes have been obtained, a determination of a significant edge is separately made for each of the image planes at 34 to identify, for example, a significant edge along or for one side of an ROI box (such as a top or upper side of the ROI box as viewed in the illustrated images). For example, a significant edge along an upper end of the ROI box may be determined such that one side of the ROI box is automatically adjusted, which may affect the height of the ROI box, as well as the curvature of the side. It should be noted that in various embodiments the width of the ROI box remains unchanged. However, in general any one or more of the sides of the ROI box may be adjusted (e.g., adjusting position and curvature) using the method 30.

With respect to the determination of the significant edge, some embodiments perform a pixel by pixel analysis for each pixel along the edge of the ROI box and moving inward from the edge to determine a first significant edge. The first significant edge may be defined as the border between two pixels wherein one pixel is a bright pixel and one pixel is a dark pixel. The bright and dark pixels may be defined by predetermined brightness threshold values (e.g., brightness levels), such that a bright pixel generally corresponds to a tissue pixel (e.g., a pixel corresponding to imaged uterine tissue) and a dark pixel generally correspond to a fluid pixel (e.g., a pixel corresponding to imaged amniotic fluid). For example, an active contour method may be performed that may also include filtering of the images. In particular, the first row of pixels along the ROI box edge is analyzed to ensure that each is a bright pixel, namely a tissue pixel. If any one of the pixels is not an imaged tissue pixel, the staring pixel row or the starting pixel may be adjusted, which may be performed automatically or manually by a user moving the ROI box or moving the side of the ROI box. Thus, for example referring to FIG. 2, the active contour method may begin at a first row of pixels adjacent an edge of the ROI boxes 68 and 70, which may be the first row of pixels along borders 69 and 71 of the ROI boxes 68 and 70, respectively. It should be noted that in various embodiments the pixels in an entire row (e.g., from the left border of the ROI box to the right border of the ROI box, namely across the width) are analyzed for a transition from a bright pixel to a dark pixel. If a transition is identified from a bright pixel to a dark pixel, the pixel(s) are marked as the first significant edge for use in defining a contour.

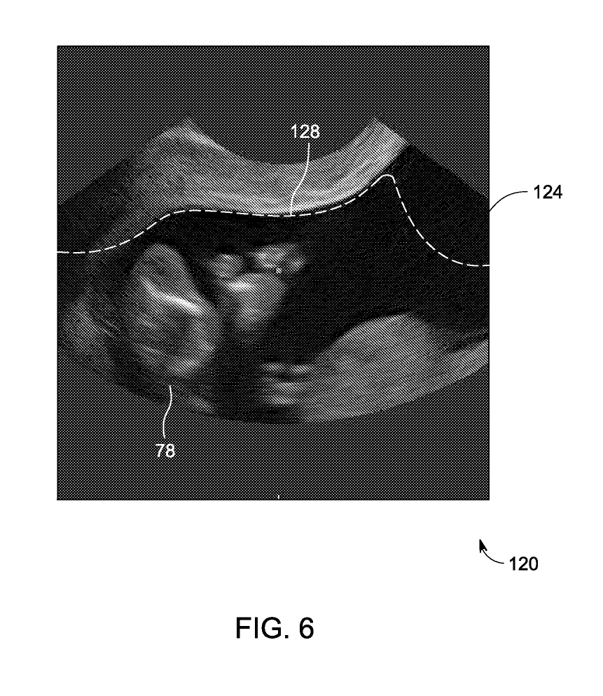

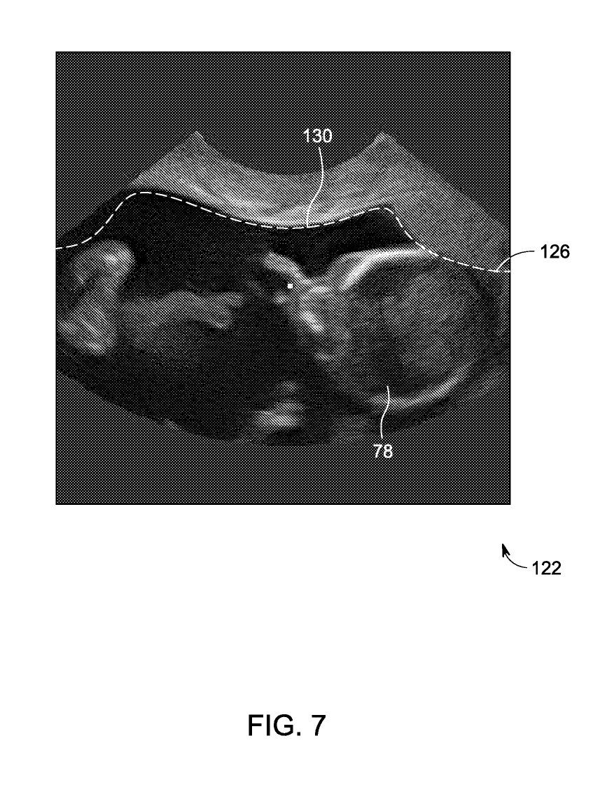

Accordingly, as illustrated in the images 120 and 122 of FIGS. 6 and 7, respectively, a contour is identified for each of the images 120 and 122 corresponding to the first significant edge pixel transition. The images 120 and 122 correspond to orthogonal image planes of the fetus 78. As can be seen, using the active contour method, a contour line 124 and 126 is separately identified for each of the images 120 and 122, respectively. The contour lines 124 and 126 generally define the boundary between tissue and fluid in the images 120 and 122. The contour lines 124 and 126 generally define a boundary for the ROI, outside of which the image should not be rendered. It should be noted that filtering to reduce noise in the images also may be performed.

Referring again to the method 30 of FIG. 1, once a contour line has been separately (or independently) determined in each of the images, the significant edge defined by the contour line in each of the images is compared at 36. For example, a determination is made for consistency, such as to determine whether the two contours have approximately the same contour and/or curvature. In some embodiments, a central point along each of the contour lines is compared to determine at 38 if the pixel corresponding to each of the center points is at approximately the same location, such as within a predetermined deviation (e.g., within 10% or within a certain number of pixels) of each other. Thus, as illustrated in FIGS. 6 and 7, central points 128 and 130 of contour lines 124 and 126, respectively, are compared to determine if the position of each is approximately the same. For example, a determination may be made as to whether the central points 128 and 130 are about the same distance (e.g., number of pixels) from the original border of the ROI box, such that the central points 128 and 130 are at about the same height.

If a determination is made at 38 that the central points are not at approximately the same location, such as the same height or distance from the original ROI box border, then at 40, the ROI is not adjusted or defined. Thus, the ROI box border is not moved or changed in contour. A user may then, for example, move the ROI box or border and initiate the method 30 again. It should be noted that the method 30, including the adjustment or defining of the ROI box that is performed automatically using the method 30 may be initiated by a user depressing a button (e.g., an ROI box adjustment button) on a user interface of the ultrasound system.

If a determination is made at 38 that the central points are at approximately same location, such as approximately the same height or distance from the original ROI box border, then a curve is fit to the contour lines at 42. For example, for each point (e.g., for each pixel) along the contour lines, a minimal distance determination may be made to fit a curve to the contour lines. In various embodiments, this determination is dependent upon the contour lines for both image planes. For example, the distance determination may be made based upon an average of the contour lines. Accordingly, the final border for the edge of the ROI box will have the same height for each of the image planes. It should be noted that optionally at 44 the ROI may be shifted or zoomed in or out based on the size of the object. For example, the ROI may be adjusted such that the ROI is not too small for the object of interest. In some embodiments the ROI box may be moved and enlarged to fit the particular user interface and display.

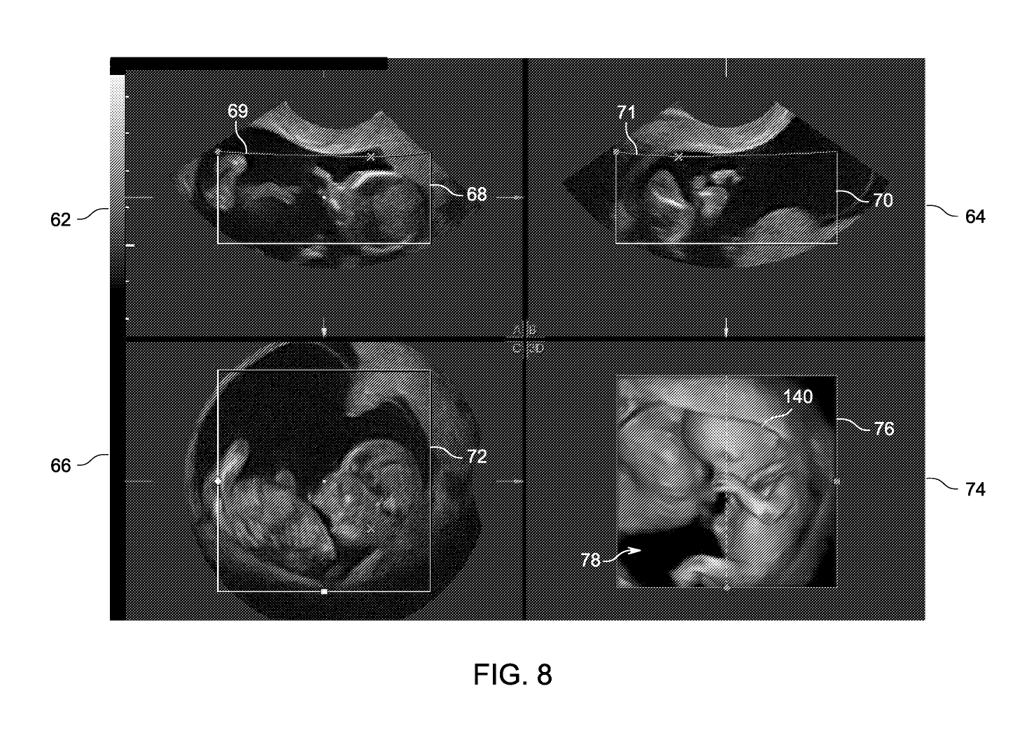

Thus, based on the fitted curves, a border for one edge of the ROI box is defined in each of the image planes and displayed at 46. Accordingly, as shown in FIG. 8, the borders 69 and 71 of the ROI boxes 68 and 70, respectively are adjusted automatically. As can bee seen, the curve that was fit to the borders 69 and 71 resulted in a curved contour that was moved downward (in FIG. 8 compared to FIG. 2). The height and curvature of each of the borders 69 and 71 is the same. The “x” along the borders 69 and 71 defines the apex of the curvature showing the point of most change along the borders 69 and 71. Thus, in various embodiments, a smooth line is fit to the determined border and includes a single control point (the “x”) along the line.

Thereafter, a determination may be made at 48 as to whether a user adjustment is made. For example, a user may determine from a visual inspection that the ROI box may need to be moved or repositioned, the border moved more, the curvature of the border changed (e.g., by dragging the “x” mark), etc. This determination may be made before or after a rendered image is generated based on the ROI box with the automatically determined border. Thus, if no user adjustment is made, then at 50 the image of the ROI is rendered based on the automatic adjustment of the one border of the ROI box. If a user adjustment is made, then the image of the ROI is rendered or re-rendered at 52 based on the used adjusted ROI box.

Thus, as illustrated in FIG. 8, the image 74 is a rendered image of the ROI defined by the ROI box 68, 70 and 72, which corresponds to ROI box 76 and having the automatically adjusted border. As can be seen in the 3D rendered image of a fetus 78, the particular area of interest, in this case a face 140 of the fetus 78, is visible and no longer obstructed by rendered tissue. Accordingly, a user is able to view the face 140 of the fetus 78 based on an automatically determined border for the ROI box.

It should be noted that the various embodiments are not limited to the particular contour detection methods described herein. In particular, the method 30 may implement any suitable method, for example, to identify the border between tissue and fluid and then fit a curve to a contour defined by the identified border. The method generally determines tissue that should not be rendered such that an ROI or particular area of interest is displayed to the user without, for example, rendered obstructing tissue.

Accordingly, various embodiments determine at least one border of an ROI, which may adjust a border of the ROI. A user thereafter may also manually adjust the ROI or border thereof. The determined border, which is determined automatically in various embodiments, results in rendered images having less or reduced obstructing pixels, for example, tissue rendered that obstructs an area of interest, such as a face of a fetus.

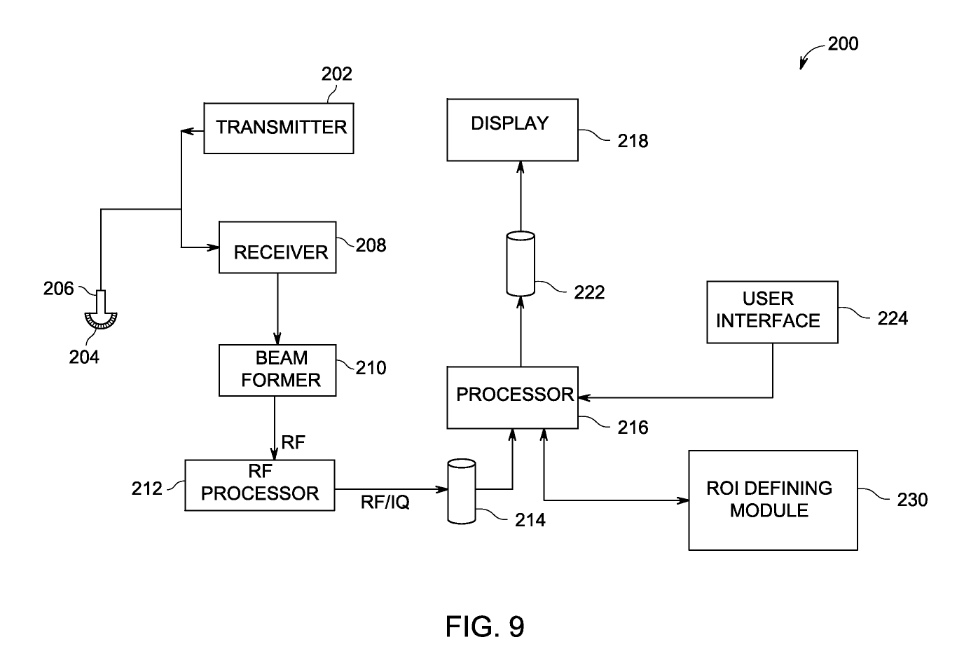

Various embodiments, including the method 30 may be implemented in an ultrasound system 200 as shown in FIG. 9, which is a block diagram the ultrasound system 200 constructed in accordance with various embodiments of the invention. The ultrasound system 200 is capable of electrical or mechanical steering of a soundbeam (such as in 3D space) and is configurable to acquire information (e.g., image slices) corresponding to a plurality of 2D representations or images of a region of interest (ROI) in a subject or patient, which may be defined or adjusted as described in more detail herein. The ultrasound system 200 is configurable to acquire 2D images in one or more planes of orientation.

The ultrasound system 200 includes a transmitter 202 that, under the guidance of a beamformer 210, drives an array of elements 204 (e.g., piezoelectric elements) within a probe 206 to emit pulsed ultrasonic signals into a body. A variety of geometries may be used. The ultrasonic signals are back-scattered from structures in the body, like blood cells or muscular tissue, to produce echoes that return to the elements 204. The echoes are received by a receiver 208. The received echoes are passed through the beamformer 210, which performs receive beamforming and outputs an RF signal. The RF signal then passes through an RF processor 212. Alternatively, the RF processor 212 may include a complex demodulator (not shown) that demodulates the RF signal to form IQ data pairs representative of the echo signals. The RF or IQ signal data may then be routed directly to a memory 214 for storage.

In the above-described embodiment, the beamformer 210 operates as a transmit and receive beamformer. In an alternative embodiment, the probe 206 includes a 2D array with sub-aperture receive beamforming inside the probe. The beamformer 210 may delay, apodize and sum each electrical signal with other electrical signals received from the probe 206. The summed signals represent echoes from the ultrasound beams or lines. The summed signals are output from the beamformer 210 to an RF processor 212. The RF processor 212 may generate different data types, e.g. B-mode, color Doppler (velocity/power/variance), tissue Doppler (velocity), and Doppler energy, for multiple scan planes or different scanning patterns. For example, the RF processor 212 may generate tissue Doppler data for multi-scan planes. The RF processor 212 gathers the information (e.g. I/Q, B-mode, color Doppler, tissue Doppler, and Doppler energy information) related to multiple data slices and stores the data information, which may include time stamp and orientation/rotation information, in the memory 214.

The ultrasound system 200 also includes a processor 216 to process the acquired ultrasound information (e.g., RF signal data or IQ data pairs) and prepare frames of ultrasound information for display on display 218. The processor 216 is adapted to perform one or more processing operations according to a plurality of selectable ultrasound modalities on the acquired ultrasound data. Acquired ultrasound data may be processed and displayed in real-time during a scanning session as the echo signals are received. Additionally or alternatively, the ultrasound data may be stored temporarily in memory 214 during a scanning session and then processed and displayed in an off-line operation.

The processor 216 is connected to a user interface 224 that may control operation of the processor 216 as explained below in more detail. A display 218 includes one or more monitors that present patient information, including diagnostic ultrasound images to the user for diagnosis and analysis. One or both of memory 214 and memory 222 may store two-dimensional (2D) or three-dimensional (3D) data sets of the ultrasound data, where such 2D and 3D data sets are accessed to present 2D (and/or 3D images). The images may be modified and the display settings of the display 218 also manually adjusted using the user interface 224.

An ROI defining module 230 is also provided and connected to the processor 216. In some embodiments, the ROI defining module 230 may be software running on the processor 216 or hardware provided as part of the processor 216. The ROI defining module 230 defines or adjusts and ROI, for example, an ROI box as described in more detail herein.

It should be noted that although the various embodiments may be described in connection with an ultrasound system, the methods and systems are not limited to ultrasound imaging or a particular configuration thereof. The various embodiments may be implemented in connection with different types of imaging systems, including, for example, x-ray imaging systems, magnetic resonance imaging (MRI) systems, computed-tomography (CT) imaging systems, positron emission tomography (PET) imaging systems, or combined imaging systems, among others. Further, the various embodiments may be implemented in non-medical imaging systems, for example, non-destructive testing systems such as ultrasound weld testing systems or airport baggage scanning systems.

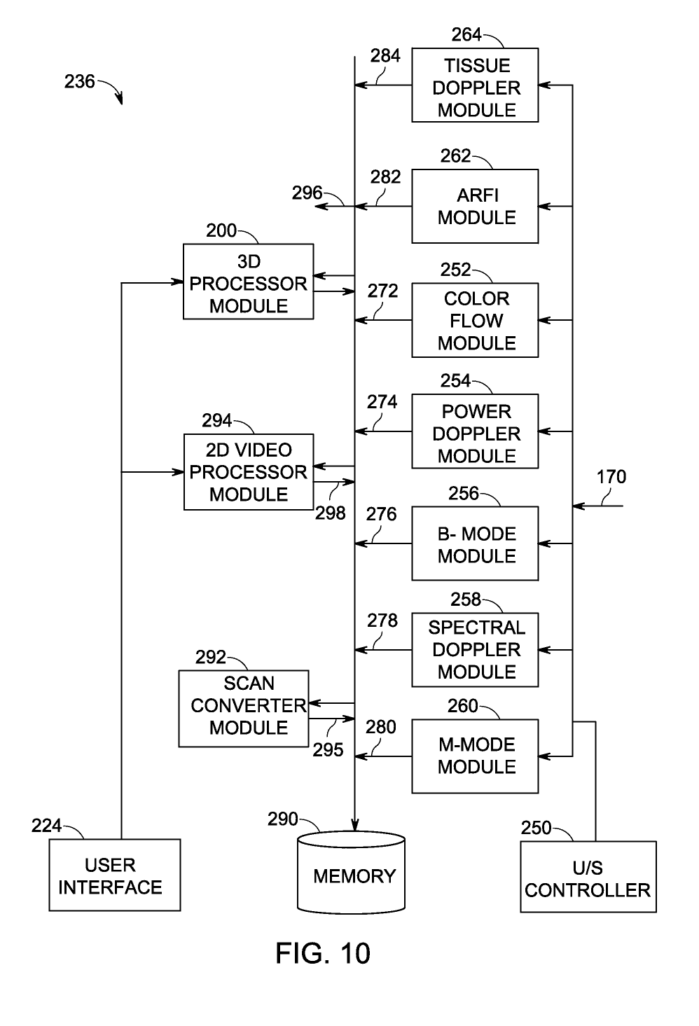

FIG. 10 illustrates an exemplary block diagram of an ultrasound processor module 236, which may be embodied as the processor 216 of FIG. 9 or a portion thereof. The ultrasound processor module 136 is illustrated conceptually as a collection of sub-modules, but may be implemented utilizing any combination of dedicated hardware boards, DSPs, processors, etc. Alternatively, the sub-modules of FIG. 10 may be implemented utilizing an off-the-shelf PC with a single processor or multiple processors, with the functional operations distributed between the processors. As a further option, the sub-modules of FIG. 10 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 10 may be controlled by a local ultrasound controller 250 or by the processor module 236. The sub-modules 252-264 perform mid-processor operations. The ultrasound processor module 236 may receive ultrasound data 270 in one of several forms. In the embodiment of FIG. 10, the received ultrasound data 270 constitutes I,Q data pairs representing the real and imaginary components associated with each data sample. The I,Q data pairs are provided to one or more of a color-flow sub-module 252, a power Doppler sub-module 254, a B-mode sub-module 256, a spectral Doppler sub-module 258 and an M-mode sub-module 260. Optionally, other sub-modules may be included such as an Acoustic Radiation Force Impulse (ARFI) sub-module 262 and a Tissue Doppler (TDE) sub-module 264, among others.

Each of sub-modules 252-264 are configured to process the I,Q data pairs in a corresponding manner to generate color-flow data 272, power Doppler data 274, B-mode data 276, spectral Doppler data 278, M-mode data 280, ARFI data 282, and tissue Doppler data 284, all of which may be stored in a memory 290 (or memory 214 or memory 222 shown in FIG. 9) temporarily before subsequent processing. For example, the B-mode sub-module 256 may generate B-mode data 276 including a plurality of B-mode image planes, such as in a triplane image acquisition as described in more detail herein.

The data 272-284 may be stored, for example, as sets of vector data values, where each set defines an individual ultrasound image frame. The vector data values are generally organized based on the polar coordinate system.

A scan converter sub-module 292 accesses and obtains from the memory 290 the vector data values associated with an image frame and converts the set of vector data values to Cartesian coordinates to generate an ultrasound image frame 295 formatted for display. The ultrasound image frames 295 generated by the scan converter module 292 may be provided back to the memory 290 for subsequent processing or may be provided to the memory 214 or the memory 222.

Once the scan converter sub-module 292 generates the ultrasound image frames 295 associated with, for example, B-mode image data, and the like, the image frames may be restored in the memory 290 or communicated over a bus 296 to a database (not shown), the memory 214, the memory 222 and/or to other processors.

The scan converted data may be converted into an X,Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the display 218 (shown in FIG. 9), which may include one or more monitors or windows of the display, to display the image frame. The image displayed in the display 218 is produced from image frames of data in which each datum indicates the intensity or brightness of a respective pixel in the display.

Referring again to FIG. 10, a 2D video processor sub-module 294 combines one or more of the frames generated from the different types of ultrasound information. For example, the 2D video processor sub-module 294 may combine a different image frames by mapping one type of data to a grey map and mapping the other type of data to a color map for video display. In the final displayed image, color pixel data may be superimposed on the grey scale pixel data to form a single multi-mode image frame 298 (e.g., functional image) that is again re-stored in the memory 290 or communicated over the bus 296. Successive frames of images may be stored as a cine loop in the memory 290 or memory 222 (shown in FIG. 9). The cine loop represents a first in, first out circular image buffer to capture image data that is displayed to the user. The user may freeze the cine loop by entering a freeze command at the user interface 224. The user interface 224 may include, for example, a keyboard and mouse and all other input controls associated with inputting information into the ultrasound system 200 (shown in FIG. 9).

A 3D processor sub-module 300 is also controlled by the user interface 224 and accesses the memory 290 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

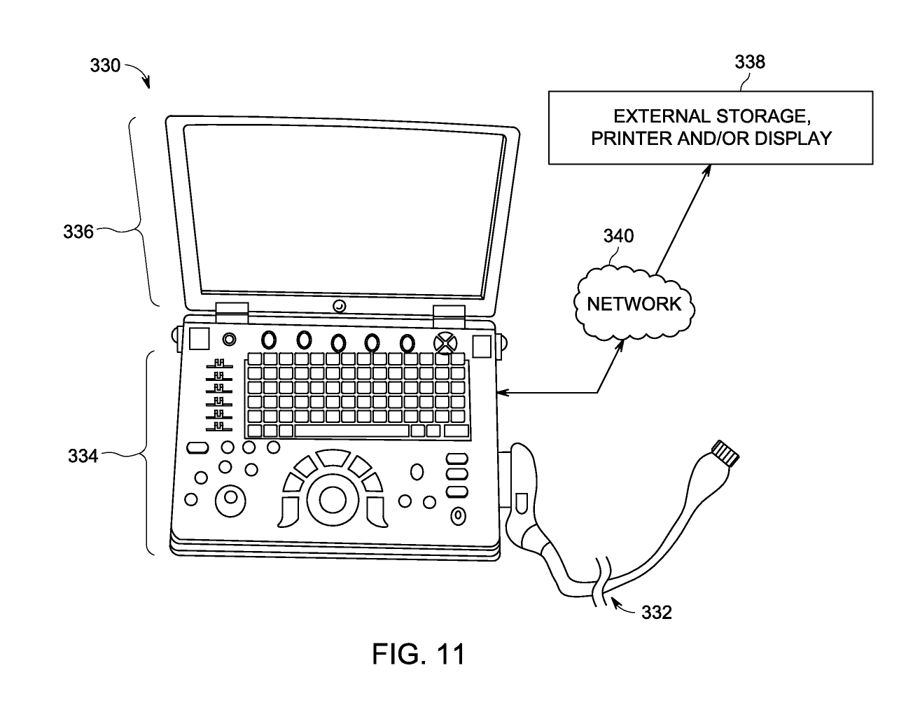

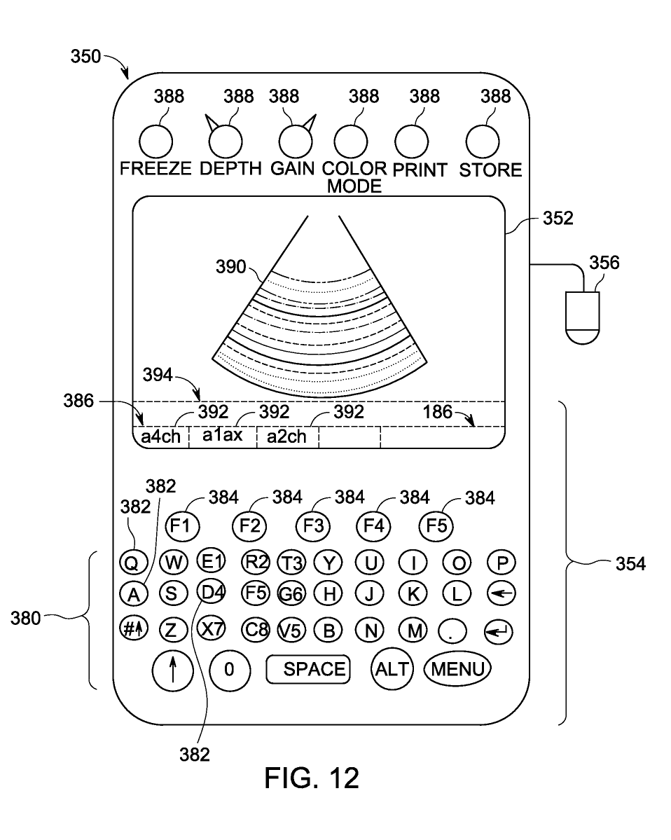

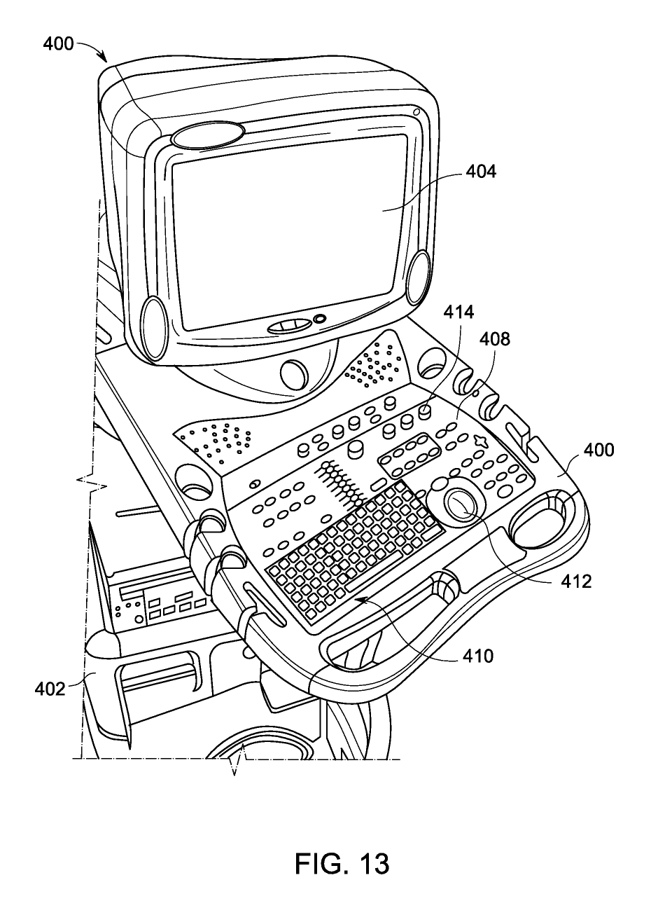

The ultrasound system 200 of FIG. 9 may be embodied in a small-sized system, such as laptop computer or pocket sized system as well as in a larger console-type system. FIGS. 11 and 12 illustrate small-sized systems, while FIG. 13 illustrates a larger system.

FIG. 11 illustrates a 3D-capable miniaturized ultrasound system 330 having a probe 332 that may be configured to acquire 3D ultrasonic data or multi-plane ultrasonic data. For example, the probe 332 may have a 2D array of elements 104 as discussed previously with respect to the probe 106 of FIG. 9. A user interface 334 (that may also include an integrated display 336) is provided to receive commands from an operator. As used herein, “miniaturized” means that the ultrasound system 330 is a handheld or hand-carried device or is configured to be carried in a person's hand, pocket, briefcase-sized case, or backpack. For example, the ultrasound system 330 may be a hand-carried device having a size of a typical laptop computer. The ultrasound system 330 is easily portable by the operator. The integrated display 336 (e.g., an internal display) is configured to display, for example, one or more medical images.

The ultrasonic data may be sent to an external device 338 via a wired or wireless network 340 (or direct connection, for example, via a serial or parallel cable or USB port). In some embodiments, the external device 338 may be a computer or a workstation having a display, or the DVR of the various embodiments. Alternatively, the external device 338 may be a separate external display or a printer capable of receiving image data from the hand carried ultrasound system 330 and of displaying or printing images that may have greater resolution than the integrated display 336.

FIG. 12 illustrates a hand carried or pocket-sized ultrasound imaging system 350 wherein the display 352 and user interface 354 form a single unit. By way of example, the pocket-sized ultrasound imaging system 350 may be a pocket-sized or hand-sized ultrasound system approximately 2 inches wide, approximately 4 inches in length, and approximately 0.5 inches in depth and weighs less than 3 ounces. The pocket-sized ultrasound imaging system 350 generally includes the display 352, user interface 354, which may or may not include a keyboard-type interface and an input/output (I/O) port for connection to a scanning device, for example, an ultrasound probe 356. The display 352 may be, for example, a 320×320 pixel color LCD display (on which a medical image 390 may be displayed). A typewriter-like keyboard 380 of buttons 382 may optionally be included in the user interface 354.

Multi-function controls 384 may each be assigned functions in accordance with the mode of system operation (e.g., displaying different views). Therefore, each of the multi-function controls 384 may be configured to provide a plurality of different actions. Label display areas 386 associated with the multi-function controls 384 may be included as necessary on the display 352. The system 350 may also have additional keys and/or controls 388 for special purpose functions, which may include, but are not limited to “freeze,” “depth control,” “gain control,” “color-mode,” “print,” and “store.”

One or more of the label display areas 386 may include labels 392 to indicate the view being displayed or allow a user to select a different view of the imaged object to display. The selection of different views also may be provided through the associated multi-function control 384. The display 352 may also have a textual display area 394 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 350 and the miniaturized ultrasound system 330 may provide the same scanning and processing functionality as the system 200 (shown in FIG. 9).

FIG. 12 illustrates an ultrasound imaging system 400 provided on a movable base 402. The portable ultrasound imaging system 400 may also be referred to as a cart-based system. A display 404 and user interface 406 are provided and it should be understood that the display 404 may be separate or separable from the user interface 406. The user interface 406 may optionally be a touchscreen, allowing the operator to select options by touching displayed graphics, icons, and the like.

The user interface 406 also includes control buttons 408 that may be used to control the portable ultrasound imaging system 400 as desired or needed, and/or as typically provided. The user interface 406 provides multiple interface options that the user may physically manipulate to interact with ultrasound data and other data that may be displayed, as well as to input information and set and change scanning parameters and viewing angles, etc. For example, a keyboard 410, trackball 412 and/or multi-function controls 414 may be provided.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, the embodiments are by no means limiting and are exemplary embodiments. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for modifying a region of interest (ROI) in an ultrasound data set, the method comprising:
defining an ROI within an acquired ultrasound data set;
identifying a plurality of different image planes within the acquired ultrasound data set;
determining a significant edge from at least one border of the ROI based on the plurality of image planes; and
adjusting the ROI based on the determined significant edge.

2. A method in accordance with claim 1 wherein determining the significant edge comprises identifying a border corresponding to a change from a bright pixel to a dark pixel.

3. A method in accordance with claim 2 wherein each of the bright pixel and dark pixel are defined by a predetermined brightness level.

4. A method in accordance with claim 1 wherein determining a significant edge is performed across a row of pixels and on a pixel by pixel basis.

5. A method in accordance with claim 1 wherein determining a significant edge comprises identifying a border corresponding to a change from a tissue pixel to a fluid pixel.

6. A method in accordance with claim 1 wherein determining a significant edge is performed separately for each of the plurality of image planes.

7. A method in accordance with claim 6 further comprising determining whether the significant edges for each of the plurality of image planes are at approximately the same location.

8. A method in accordance with claim 1 further comprising fitting a curve to the determined significant edge.

9. A method in accordance with claim 8 wherein the curve fitting is based on a least distance determination from a contour defined by the determined significant edge.

10. A method in accordance with claim 1 wherein the ROI is defined by an ROI box and the adjusting comprises changing at least one of a height or curvature of one border of the ROI box.

11. A method in accordance with claim 1 further comprising changing one of a position or zoom level of the adjusted ROI.

12. A method in accordance with claim 1 further comprising receiving a user input and changing the adjusted ROI based on the received user input.

13. A method in accordance with claim 1 wherein the ROI is defined by an ROI box and wherein a width of the ROI box remains unchanged.

14. A method in accordance with claim 1 wherein the plurality of image planes comprise at least two orthogonal image planes.

15. A method in accordance with claim 1 wherein the ultrasound data set corresponds to an imaged fetus.

16. A method for adjusting a region of interest (ROI) in an ultrasound data set, the method comprising:
determining an ROI based on an ROI box defined within at least two image planes, the ROI box having a width, height and depth;
identifying pixels from a top side of the ROI box that define a border wherein pixels change from tissue pixels to fluid pixels;
fitting a curve to a contour based on the border; and
adjusting the height of the ROI box based on the fitted curve.

17. A method in accordance with claim 16 further comprising adjusting a curvature of the top side of the ROI box.

18. A method in accordance with claim 16 wherein the tissue pixel corresponds to imaged uterine tissue and the fluid pixel corresponds to imaged amniotic fluid.

19. A method in accordance with claim 16 wherein the pixels defining the border are identified separately for each of the plurality of image planes.

20. An ultrasound system comprising:
an ultrasound probe for acquiring ultrasound data for an object of interest;
a user interface for defining a region of interest (ROI) within at least two different image planes within the ultrasound data; and
an ROI defining module configured to adjust an ROI based on a determination of a significant edge from at least one border of the ROI based on the two image planes.
